(19) United States
(12) Patent Application Publication
(10) Pub. No.: US 2003/0186301 A1
(43) Pub. Date: Oct. 2, 2003

(54) Constructing user-defined, long DNA sequences

(75) Inventors: Allen T. Christian, Tracy, CA (US); Raymond P. Mariella JR., Danville, CA (US); James D. Tucker, Novi, MI (US)

Correspondence Address: Eddie E. Scott, Assistant Laboratory Counsel, Lawrence Livermore National Laboratory, P.O. Box 808, L-703, Livermore, CA, 94551, US

(73) Assignee: The Regents of the University of California

(21) Appl. No.: 10/393,614

(22) Filed: Mar. 19, 2003

Related U.S. Application Data
(60) Provisional application No. 60/367,929, filed on Mar. 25, 2002.
(60) Provisional application No. 60/367,928, filed on Mar. 25, 2002.

(51) Int. Cl. C12Q 1/68; C07H 21/04; C12P 19/34

(52) U.S. Cl. 435/6; 435/91.2; 536/25.3

(57) ABSTRACT

A system for synthesizing a DNA molecule of user-defined sequence. A pre-defined, double-stranded, sequence of DNA with a single-stranded overhang is provided. The pre-defined, double-stranded, sequence of DNA is lengthened by the addition user-selected, single stranded DNA sequences.

### CROSS-REFERENCE TO RELATED APPLICATIONS

This application claims the benefit of U.S. Provisional Patent Application No. 60/367,929 filed Mar. 25, 2002 titled “Voltage-Controlled Method for Constructing User-Defined, Very Long DNA Sequences” and U.S. Provisional Patent Application No. 60/367,928 filed Mar. 25, 2002 titled “Method for Constructing User-Defined, Very Long DNA Sequences.” U.S. Provisional Patent Application No. 60/367,929 filed Mar. 25, 2002 titled “Voltage-Controlled Method for Constructing User-Defined, Very Long DNA Sequences” and U.S. Provisional Patent Application No. 60/367,928 filed Mar. 25, 2002 titled “Method for Constructing User-Defined, Very Long DNA Sequences” are incorporated in this application by this reference.

### CONSTRUCTING USER-DEFINED, LONG DNA SEQUENCES

The United States Government has rights in this invention pursuant to Contract No. W-7405-ENG-48 between the United States Department of Energy and the University of California for the operation of Lawrence Livermore National Laboratory.

### BACKGROUND

1. Field of Endeavor

The present invention relates to DNA sequences and more particularly to constructing long DNA sequences.

2. State of Technology

U.S. Pat. No. 6,375,903 issued Apr. 23, 2002 to Francesco Cerrina, Michael R. Sussman, Frederick R. Blattner, Sangeet Singh-Gasson, and Roland Green for a method and apparatus for synthesis of arrays of DNA probes assigned to the Wisconsin Alumni Research Foundation provides the following background information:

“The sequencing of deoxyribonucleic acid (DNA) is a fundamental tool of modern biology and is conventionally carried out in various ways, commonly by processes which separate DNA segments by electrophoresis. See, e.g., Current Protocols In Molecular Biology, Vol. 1, Chapter 7, “DNA Sequencing,” 1995. The sequencing of several important genomes has already been completed (e.g., yeast, E. coli), and work is proceeding on the sequencing of other genomes of medical and agricultural importance (e.g., human, C. elegans, Arabidopsis). In the medical context, it will be necessary to “re-sequence” the genome of large numbers of human individuals to determine which genotypes are associated with which diseases. Such sequencing techniques can be used to determine which genes are active and which inactive either in specific tissues, such as cancers, or more generally in individuals exhibiting genetically influenced diseases. The results of such investigations can allow identification of the proteins that are good targets for new drugs or identification of appropriate genetic alterations that may be effective in genetic therapy. Other applications lie in fields such as soil ecology or pathology where it would be desirable to be able to isolate DNA from any soil or tissue sample and use probes from ribosomal DNA sequences from all known microbes to identify the microbes present in the sample.

The conventional sequencing of DNA using electrophoresis is typically laborious and time consuming. Various alternatives to conventional DNA sequencing have been proposed. One such alternative approach, utilizing an array of oligonucleotide probes synthesized by photolithographic techniques is described in Pease, et al., “Light-Generated Oligonucleotide Arrays for Rapid DNA Sequence Analysis,” Proc. Natl. Acad. Sci. USA, Vol. 91, pp. 5022-5026, May 1994. In this approach, the surface of a solid support modified with photolabile protecting groups is illuminated through a photolithographic mask, yielding reactive hydroxyl groups in the illuminated regions. A 3′ activated deoxynucleoside, protected at the 5′ hydroxyl with a photolabile group, is then provided to the surface such that coupling occurs at sites that had been exposed to light. Following capping, and oxidation, the substrate is rinsed and the surface is illuminated through a second mask to expose additional hydroxyl groups for coupling. A second 5′ protected activated deoxynucleoside base is presented to the surface. The selective photodeprotection and coupling cycles are repeated to build up levels of bases until the desired set of probes is obtained. It may be possible to generate high density miniaturized arrays of oligonucleotide probes using such photolithographic techniques wherein the sequence of the oligonucleotide probe at each site in the array is known. These probes can then be used to search for complementary sequences on a target strand of DNA, with detection of the target that has hybridized to particular probes accomplished by the use of fluorescent markers coupled to the targets and inspection by an appropriate fluorescence scanning microscope. A variation of this process using polymeric semiconductor photoresists, which are selectively patterned by photolithographic techniques, rather than using photolabile 5′ protecting groups, is described in McGall, et al., “Light-Directed Synthesis of High-Density Oligonucleotide Arrays Using Semiconductor Photoresists,” Proc. Natl. Acad. Sci. USA, Vol. 93, pp. 13555-13560, November 1996, and G. H. McGall, et al., “The Efficiency of Light-Directed Synthesis of DNA Arrays on Glass Substrates,” Journal of the American Chemical Society 119, No. 22, 1997, pp. 5081-5090.

A disadvantage of both of these approaches is that four different lithographic masks are needed for each monomeric base, and the total number of different masks required are thus four times the length of the DNA probe sequences to be synthesized. The high cost of producing the many precision photolithographic masks that are required, and the multiple processing steps required for repositioning of the masks for every exposure, contribute to relatively high costs and lengthy processing times.”

International Patent Application WO 02/04597 by Roland Green and Nicholas, J. Seay published Jan. 17, 2002 and assigned to Nimblegen Systems, Inc. for a method and apparatus for the synthesis of arrays of DNA probes provides the following background information:

“ . . . the synthesis of arrays of DNA probe sequences, polypeptides, and the like is carried out rapidly and efficiently using patterning processes. The process may be automated and computer controlled to allow the fabrication of a one or two-dimensional array of probes containing probe sequences customized to a particular investigation. No lithographic masks are required, thus eliminating the significant costs and time delays associated with the production of lithographic masks and avoiding time-consuming manipulation and alignment of multiple masks during the fabrication process of the probe affays.”

International Patent Application WO 02/095073 by Peter J. Belshaw, Michael, R. Sussman, and Francesco Cerrina published Nov. 28, 2002 and assigned to the Wisconsin Alumni Research Foundation for a method for the synthesis of DNA sequences provides the following background information:

“Using the techniques of recombinant DNA chemistry, it is now common for DNA sequences to be replicated and amplified from nature and for those sequences to then be disassembled into component parts which are then recombined or reassembled into new DNA sequences. While it is now both possible and common for short DNA sequences, referred to as oligonucleotides, to be directly synthesized from individual nucleosides, it has been thought to be generally impractical to directly construct large segments or assemblies of DNA sequences larger than about 400 base pairs. As a consequence, larger segments of DNA are generally constructed from component parts and segments which can be purchased, cloned or synthesized individually and then assembled into the DNA molecule desired. The present invention is summarized in a method for constructing a DNA construct of defined sequence. The method begins with breaking up the sequence into a plurality of overlapping DNA segments using computer software. A DNA microarray is then made on a substrate in such a way that each single stranded probe on the array is constructed to be one of the overlapping DNA segments needed to make up the desired DNA construct. Then the probes are all released from the substrate. The probes will then self assemble into the desired DNA construct.”

### SUMMARY

Features and advantages of the present invention will become apparent from the following description. Applicants are providing this description, which includes drawings and examples of specific embodiments, to give a broad representation of the invention. Various changes and modifications within the spirit and scope of the invention will become apparent to those skilled in the art from this description and by practice of the invention. The scope of the invention is not intended to be limited to the particular forms disclosed and the invention covers all modifications, equivalents, and alternatives falling within the spirit and scope of the invention as defined by the claims.

The present invention provides a system for synthesizing a DNA molecule of user-defined sequence. A pre-defined, double-stranded, sequence of DNA with a single-stranded overhang is provided. The pre-defined, double-stranded, sequence of DNA is lengthened by the addition user-selected, single stranded DNA sequences.

The invention is susceptible to modifications and alternative forms. Specific embodiments are shown by way of example. It is to be understood that the invention is not limited to the particular forms disclosed. The invention covers all modifications, equivalents, and alternatives falling within the spirit and scope of the invention as defined by the claims.

### BRIEF DESCRIPTION OF THE DRAWINGS

The accompanying drawings, which are incorporated into and constitute a part of the specification, illustrate specific embodiments of the invention and, together with the general description of the invention given above, and the detailed description of the specific embodiments, serve to explain the principles of the invention.

FIG. 1 illustrates the beginning of the synthesis of a DNA molecule with a surface-tethered, pre-defined, double-stranded, sequences of DNA approximately 30 base pairs long with a short, single-stranded overhang.

FIG. 2 illustrates an oligo of six bases used as the user-selected, single stranded DNA sequence.

FIG. 3 illustrates the selected oligo annealing to the initial DNA sequence by way of hydrogen bonding to the overhanging strand, thereby generating a new overhang.

FIG. 4 illustrates process being repeated with additional oligos until the desired full-length DNA sequence has been constructed.

FIG. 5 illustrates use of a pre-defined double-stranded sequence of approximately 30 base pairs in length to finish the DNA sequence.

FIG. 6 illustrates the final full-length DNA product.

### DETAILED DESCRIPTION OF THE INVENTION

Synthetic oligonucleotides are extremely valuable for molecular biology. Used for PCR, in situ hybridization, gene transfection and mutagenesis, among other things, the ability to make artificial DNA of a known sequence is absolute necessity for molecular biology. Phosphoramidite oligonucleotide synthesis is used extensively for this purpose. It has the advantage of universal application; any desired sequence can be programmed into a computer, and the machine quickly produces the sequence. This process has led to a revolution in molecular biology, but it possesses significant shortcomings. The two most significant are the size of the machine and the length of the product that it can produce. Current-generation phosphoramidite synthesis machines weigh nearly 400 pounds, and occupy a significant volume of space. While this is not a problem in most laboratory settings, certain applications for gene synthesis require a portable device.

Miniaturization could possibly overcome the size limitation of the device, but the problem of product length is more significant. The practical length limitation for phosphoramidite-synthesized oligonucleotides is approximately 120 bases. Error rates in the addition of each base preclude achieving products of significantly greater length. This length is adequate for many applications of biology, but no known genes are as short as 120 bases. The various methods for creating DNA strands longer than 120 bases use phosphoramidite synthesis as one component of the reaction, but each of the various methods requires additional technology to achieve nucleic acid lengths greater than 120 bases.

Long synthetic oligonucleotides themselves have many uses. The uses include making artificial genes for mutagenesis research and designer protein manufacturing, internal research for mutagenesis, DNA repair and carcinogenesis studies, for information storage, research on mutagenesis, DNA repair, carcinogenesis, and other fields that would benefit by the development of a rapid and simple process for gene synthesis. The effects of single base mutations on the transcription and translation of a particular gene could be studied easily if large quantities of a variety of long gene sequences could be made rapidly.

There is no easily scalable method for creating these products at the current time. A variety of ways to make artificial genes exists, but none is man-portable, and none has the ability to make any desired gene without extensive lead time. By way of comparison, the technology known as the polymerase chain reaction has been possible for more than thirty years. With three water baths of different temperatures, a scientist could move a tube of DNA from one bath to the other, adding new polymerase after each cycle through the three baths. The process took hours, and since each cycle lasted only a few minutes the labor cost was intensive, as was the cost of the non-thermostable polymerase. These costs made the early incarnations of PCR a nonviable way to replicate DNA. The discovery of thermostable DNA polymerase and the invention of the automated thermocycler made PCR so fast, inexpensive and simple that the procedure has become a common fixture from the state of the art clinical and research labs to junior high school science classes.

Applicants have developed a system for making long, double-stranded synthetic polynucleotides. This process consists of sequentially hybridizing short single-stranded oligonucleotides (oligos) to each other, followed by enzymatic ligation. This results in a contiguous piece of PCR-ready double-stranded DNA of predetermined sequence that can be extended many thousands of base pairs. Caches of the different possible DNA hexamers are synthesized by conventional phosphoramidite synthesis prior to the long poly-nucleotide synthesis, and kept in the synthesis device to be drawn upon as need to create the desired molecule. This makes the long-strand nucleotide synthesis independent of in loco phosphoramidite syntheses. Since phosphoramidite synthesis is a fairly slow process requiring expensive and bulky equipment, the ability to pre-synthesize all of the components results in a significantly streamlined process. This procedure can be used to synthesize artificial genes, DNA or RNA probes, primers or any other molecule made of ribonucleic or deoxyribonucleic acid. While each of the individual aspects of this technology existed prior to our development work, we have combined them in a novel way that makes synthetic gene assembly rapid, universal, and portable.

Some of the advantages of applicants invention are that it takes full advantage of existing and widely-used DNA synthesis technologies, it minimizes the number of enzymatic ligation reaction steps, which can number in the thousands under the first approach, and it allows development of the desired final product to proceed in stages, for example, if a 10,000 base sequence is prohibitively difficult to build, it would be possible to produce 10 sequences that are each 1000 bases long, and join them together.

The gene synthesis process can be marketed as both a service, and as an in-laboratory instrument. The process is flexible enough that gene sequences could be ordered and delivered, much like short oligonucleotides are ordered for use in PCR. For laboratories that needed greater quantities of various genes, or needed long sequences on a more frequent basis, the entire device could be sold. This would also engender a secondary market for reagents. The 4096 different hexamers necessary to make any sequence could be sold as a plug-in module, and the polymerases and ligases could also be marketed. This is similar to the way that phosphoramidite synthesis machines and DNA sequencers are marketed, both as services and as equipment.

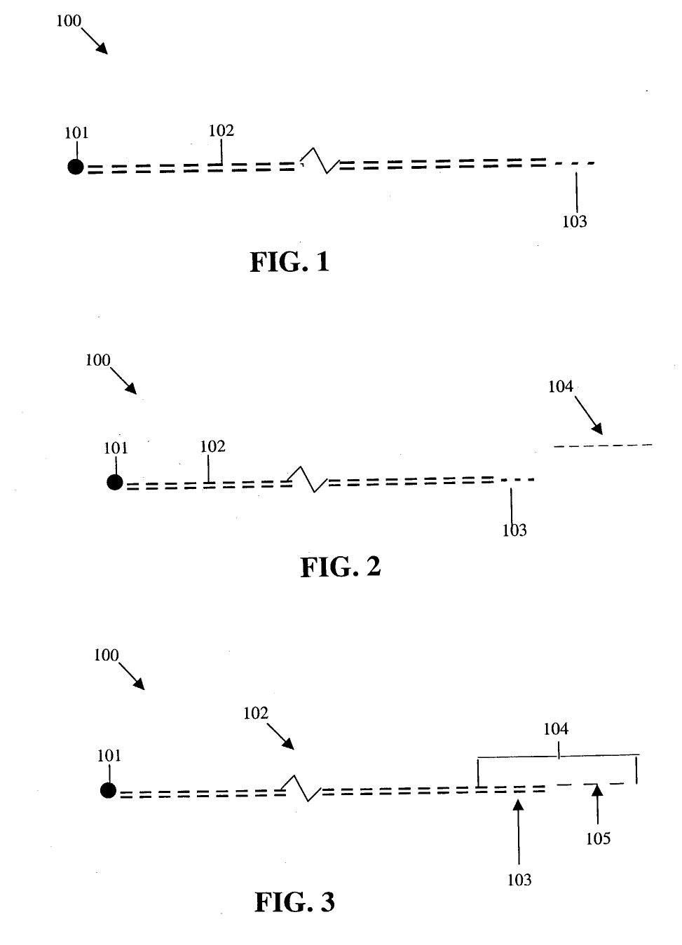

Referring now to FIGS. 1-6 of the drawings, one embodiment of the system for synthesizing a long DNA molecule in accordance with the present invention is illustrated. The full DNA molecule is designated generally by the reference numeral 100. The DNA molecule 100 is a DNA molecule of predetermined sequence. Once the specific DNA molecule that is to be synthesized has been determined, the DNA molecule is broken into segments by a computer program. The segments combined and assembled to produce the DNA molecule 100 in accordance with the present invention.

The DNA molecule can be synthesized using array technology that is known in the art. For example, U.S. Pat. No. 6,238,868, incorporated herein by reference, provides the following information, “microchip device is an electronically controlled microelectrode array. See, PCT application WO96/01836, the disclosure of which is hereby incorporated by reference. In contrast to the passive hybridization environment of most other microchip devices, the electronic microchip devices (or active microarray devices) of the present invention offer the ability to actively transport or electronically address nucleic acids to discrete locations on the surface of the microelectrode array, and to bind the addressed nucleic acid at those locations to either the surface of the microchip at specified locations.”

As illustrated in FIG., 1, the synthesis of the DNA molecule 100 begins with surface-tethered, pre-defined, double-stranded, sequences of DNA base pairs with a short, single-stranded overhang. The surface-tethered, pre-defined, double-stranded, sequences of DNA is a T7 primer 102. The T7 primer 102 is a pre-defined, double-stranded, sequences of DNA approximately 30 base pairs long with a short, single-stranded overhang. This type of primer is commercially available. The T7 primer 102 has a short, single-stranded overhang 103. The overhang 103 comprises a three bases overhang. A bead 101 is attached to the T7 primer 102. The surface is voltage controlled according to systems known in the art.

In other embodiments, instead of using a T7 primer for the pre-defined, double-stranded, sequences of DNA base pairs with a short, single-stranded overhang, different primers are used. In one of the other embodiments, the pre-defined, double-stranded, sequences of DNA base pairs with a short, single-stranded overhang is a T3 primer. The T3 primer is commercially available. The T3 primer used for primer 102 has a short, single-stranded overhang 103. The overhang 103 comprises a three bases overhang. A bead 101 is attached to the T3 primer 102. The surface is voltage controlled according to systems known in the art. In still another embodiments, the pre-defined, double-stranded, sequences of DNA base pairs with a short, single-stranded overhang is a SP6 primer. The SP6 primer is commercially available. The SP6 primer used for primer 102 has a short, single-stranded overhang 103. The overhang 103 comprises a three bases overhang. A bead 101 is attached to the SP6 primer 102. The surface is voltage controlled according to systems known in the art. In other embodiments, the pre-defined, double-stranded, sequences of DNA base pairs with a short, single-stranded overhang 102 are primers known in the art and that are to be discovered.

Construction of the full-length DNA product involves a repetitive process in which the initial DNA sequence is lengthened by the addition of a user-selected, single stranded DNA sequence, called an oligonucleotide (“oligo”) comprised of approximately 6 (or more) bases. As illustrated by FIG. 2, an oligo 104 of six bases is used as the user-selected, single stranded DNA sequence.

The selected oligo anneals to the initial DNA sequence by way of hydrogen bonding to the overhanging strand, thereby generating a new overhang. The bases at the proximal end of the oligo must, therefore, be complementary to the overhanging bases. The oligo is then covalently attached to the initial sequence using an enzyme called ligase. As illustrated by FIG. 3, the selected oligo 104 anneals to the initial DNA sequence 102 by way of hydrogen bonding to the overhanging strand 103, thereby generating a new overhang 105. The three bases at the proximal end of the oligo 104 must are complementary to the overhanging three bases 103 on the T7 primer 102. The oligo 104 is then covalently attached to the initial sequence using an enzyme called ligase.

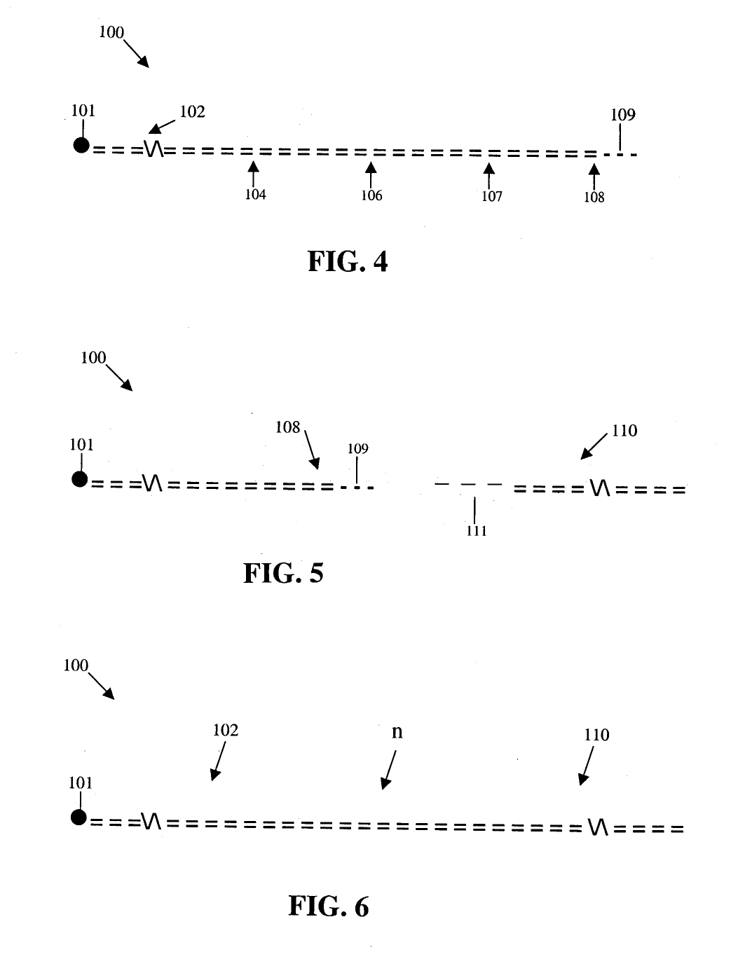

The excess oligo and ligase are removed, and the process is repeated with additional oligos until the desired full-length DNA sequence has been constructed. As illustrated by FIG. 4, the excess oligo and ligase are removed. The process is repeated with additional oligos 106, 107, 108, etc. until the desired full-length DNA sequence 100 has been constructed.

Different embodiments of the invention utilize different types of ligases and conditions. In other embodiments temperatures are used from 0 degrees C. to 70 degrees C. In other embodiments different ligases are used including T4 DNA ligase, T7 RNA ligase, and Ampligase thermostable DNA ligase.

After the last oligo 108 has been attached, the DNA sequence is finished by ligating a pre-defined double-stranded sequence approximately 30 base pairs in length, which has a single-stranded overhang complementary to the overhang of the final oligo. This 30-base-pair sequence may either be identical to or different than the first sequence that was attached to the surface.

As illustrated by FIG. 5 a pre-defined double-stranded sequence 110 approximately 30 base pairs in length is used to finish the DNA sequence 100. The sequence 110 has a single-stranded overhang complementary to the overhang 109 of the final oligo 108. The final step involves PCR amplification of the full-length sequence 100 using primers complementary to the 30-base-pair termini. The final full-length DNA product 100 is illustrated in FIG. 6. The full-length DNA product 100 comprises T7 primer 102, the preselected number of oligos n and the pre-defined double-stranded sequence 110.

In other embodiments of the present invention providing systems for synthesizing a long DNA molecule of user-defined sequence, the final DNA molecule to be synthesized is broken into segments by a computer program. The segments are combined and assembled to produce the final DNA molecule. A first pre-defined sequence of DNA is provided. The first pre-defined sequence of DNA is lengthened by sequentially combining reactions in which the first pre-defined sequence of DNA are lengthened by the addition of additional pre-defined sequences of DNA, and sequentially combining reactions in which the additional pre-defined sequences of DNA are lengthened by the addition of yet additional pre-defined sequences of DNA. In another embodiment of the present invention the steps of sequentially combining reactions in which the first pre-defined sequences of DNA are lengthened by the addition of additional pre-defined sequences of DNA and sequentially combining reactions in which the additional pre-defined sequences of DNA are lengthened by the addition of yet additional pre-defined sequences of DNA are performed in parallel.

The system relies on traditional phosphoramidite chemistries to produce medium length (ca. 100 base) sequences, which are synthesized and subsequently connected in the field to produce the desired final product. A three-step process is envisioned. In the first step, a phosphoramidite synthesizer operates in parallel on a dozen or more chambers or wells, with the final product of each series of reactions temporarily stored until needed. Each synthesizer is used to produce multiple batches of 100 base sequences until the necessary number of sequences has been produced. The second step has 4 possible scenarios, which are described below. The third and final step involves PCR amplification of the full-length product using primers complementary to the ends of the finished sequence.

Scenario 1—Sequential Enzymatic Ligation with Anchored Beads. This scenario is very similar to the first approach described above, except that the oligos added at each step are 100 bases rather than 6 bases long. The process starts with an anchored bead attached to a defined 30-base-pair sequence. Here however, instead of a 3-base overhang, a longer overhang in the range of 15-20 bases is used. The first 100-base sequence is ligated to the anchored sequence, and the excess 100-base DNA and the ligase are washed off. The process is then repeated as necessary. The final step involves annealing and ligating a sequence of the appropriate length to the long single-stranded overhang, so that the finished molecule is entirely double-stranded. Alternatively, the single-stranded portion can be filled in with endonuclease, but this would involve the use of an additional enzyme and its associated chemistries.

Scenario 2—Sequential Chemical Ligation with Anchored Beads. As in the previous embodiment, but the 100-base sequences are connected using phosphoramidite chemistries. Anoxic, anhydrous conditions need to be maintained.

Scenario 3—Un-anchored “free solution” Reactions. All of the 100-base sequences are pooled into a single reaction, along with short (−20-base) oligos that are used as latches which anneal to and hold together the termini of each adjacent pair of 100-base sequences in the appropriate final order. The latch sequences are synthesized in parallel to the 100-base sequences. Once the sequences have had sufficient time to anneal, DNA ligase are added to establish covalent bonds between the 100-base sequences. Finally, endonuclease is added to fill in the single-stranded regions between the latches, and ligase provide the final annealing of the extended latches. This approach offers considerable simplicity in the instrumentation.

Scenario 4—Nanotechnologies. Each 100-base sequence is attached to the end of one tooth of a “comb,” arrayed linearly in the order that the sequences are to be joined. Once the sequences have been attached, the tines are moved close enough together to permit ligation to occur in the desired order, but the comb's teeth are kept far enough apart to avoid generating incorrect sequences. The sequences are joined either by ligation or by phosphoramidite chemistries as described above.

There is a trade-off between the length of the oligos employed and the number of ligation reactions that need to be performed to produce the final sequence. The number of oligos that must be arrayed is a major consideration here. For 6-base oligos, 4096 (i.e., 46) combinations are possible. Arrays of this size are well within the realm of current robotic technologies. Oligos shorter than 6 bases require a slight increase in the number of ligation reactions needed, whereas longer oligos dramatically increase the number of elements required in the array. Oligos of 6 bases appear optimal, based on the number of arrayed elements that would be required and the number of ligation reactions that would be needed. However, the optimal oligo length will have to be determined experimentally. Note that oligos with an odd number of bases would produce different numbers of overhanging bases. For example, with oligos of 7 bases the first and all subsequent odd-numbered reactions would result in overhangs of 4 bases, whereas the even-number reactions would produce 3-base overhangs.

Some repetitive sequences may be difficult to produce. For example, consider the 6-base sequence 5′-AAAxyz-3′, where A is adenine, one of the 4 DNA bases, and x, y, and z represent any base. This is one possible oligo that could be added to the terminal sequence 3′- . . . TTT-5′. Note, however, that this oligo might misalign in a “staggered” way, so that only 2 rather than all 3 adenines anneal. If this happens, the ligation step will not occur. However, it is highly improbable that all of the anchored DNA strands will experience this mis-alignment problem, and those that do successfully complete each of the reactions will be amplified by PCR in the final step. Poor alignments between the oligo and the anchored strand are thermodynamically less favorable than good alignments and are expected to be relatively rare. Furthermore, unreacted ends of the growing DNA sequence will not extend efficiently, as each subsequent oligo has only a small chance of matching the overhanging three-base sequence produced by the previous step (1/64 assuming complete randomness).

Another embodiment of the present invention comprises, building long DNA with in-the-field synthesis of medium length sequences. This approach relies on traditional phosphoramidite chemistries to produce medium-length (ca. 100 base) sequences, which are synthesized and subsequently connected in the field to produce the desired final product. Applicants utilize a three-step process. In the first step, a phosphoramidite synthesizer operates in parallel on a dozen or more chambers or wells, with the final product of each series of reactions temporarily stored until needed. Each synthesizer is used to produce multiple batches of 100 base sequences until the necessary number of sequences has been produced. The second step has 4 possible scenarios, which are described below. The third and final step involves PCR amplification of the full-length product using primers complementary to the ends of the finished sequence.

Sequential Enzymatic Ligation with Anchored Beads. This embodiment is very similar to the embodiment described above and illustrated in FIGS. 1-6, except that the oligos added at each step are 100 bases rather than 6 bases long. The process starts with an anchored bead attached to a defined 30 base pair sequence. Here however, instead of a 3-base overhang, a longer overhang in the range of 15-20 bases is used. The first 100 base sequence is ligated to the anchored sequence, and the excess 100-base DNA and the ligase are washed off. The process is then repeated as necessary. The final step involves annealing and ligating a sequence of the appropriate length to the long single-stranded overhang, so that the finished molecule is entirely double-stranded. Alternatively, the single-stranded portion can be filled in with endonuclease, but this would involve the use of an additional enzyme and its associated chemistries.

Sequential Chemical Ligation with Anchored Beads. As above, but the 100-base sequences are connected using phosphoramidite chemistries. Anoxic, anhydrous conditions need to be maintained.

Un-Anchored “free solution” Reactions. All of the 100-base sequences are pooled into a single reaction, along with short (−20-base) oligos that are used as latches which anneal to and hold together the termini of each adjacent pair of 100-base sequences in the appropriate final order. The latch sequences are synthesized in parallel to the 100-base sequences. Once the sequences have had sufficient time to anneal, DNA ligase is added to establish covalent bonds between the 100-base sequences. Finally, endonuclease is added to fill in the single-stranded regions between the latches, and ligase would provide the final annealing of the extended latches.

Comb-Anchored Ligation. This embodiment utilizes nanotechnologies. Here, each 100-base sequence is attached to the end of one tooth of a “comb,” arrayed linearly in the order that the sequences are to be joined. Once the sequences have been attached, the tines are moved close enough together to permit ligation to occur in the desired order, but the comb's teeth are kept far enough apart to avoid generating incorrect sequences. The sequences may be joined either by ligation or by phosphoramidite chemistries as indicated above.

It should be understood that the invention is not intended to be limited to the particular forms disclosed. Rather, the invention is to cover all modifications, equivalents, and alternatives falling within the spirit and scope of the invention as defined by the following appended claims.

1. A method of synthesizing a DNA molecule of user-defined sequence, comprising the steps of:
providing a pre-defined, double-stranded, sequence of DNA with a single-stranded overhang, and
lengthening said pre-defined, double-stranded, sequences of DNA by the addition of user-selected, single stranded DNA sequences.

2. The method of synthesizing a DNA molecule of user-defined sequence of claim 1, including the step of tethered said pre-defined, double-stranded, sequence of DNA with a single-stranded overhang.

3. The method of synthesizing a DNA molecule of user-defined sequence of claim 1, including the step of tethered said pre-defined, double-stranded, sequence of DNA with a bead.

4. The method of synthesizing a DNA molecule of user-defined sequence of claim 1, including the step of ligating user-selected, single stranded DNA sequences to lengthen said surface-tethered, pre-defined, double-stranded, sequence of DNA.

5. The method of synthesizing a DNA molecule of user-defined sequence of claim 1, including the step of adding a final user-selected, single stranded DNA sequences creating a final overhang and ligating a pre-defined double-stranded sequence which has a single-stranded overhang complementary to said final overhang to said final user-selected, single stranded DNA sequence.

6. The method of synthesizing a DNA molecule of user-defined sequence of claim 1 wherein said user-selected, single stranded DNA sequence anneals to said pre-defined, double-stranded, sequence of DNA with a single-stranded overhang by way of hydrogen bonding to said single-stranded overhang.

7. The method of synthesizing a DNA molecule of user-defined sequence of claim 1 wherein said pre-defined, double-stranded, sequence of DNA with a single-stranded overhang is approximately 30 base pairs long.

8. The method of synthesizing a DNA molecule of user-defined sequence of claim 1 wherein said pre-defined, double-stranded, sequence of DNA with a single-stranded overhang is a T7 primer.

9. The method of synthesizing a DNA molecule of user-defined sequence of claim 1 wherein said pre-defined, double-stranded, sequence of DNA with a single-stranded overhang is a T3 primer.

10. The method of synthesizing a DNA molecule of user-defined sequence of claim 1 wherein said pre-defined, double-stranded, sequence of DNA with a single-stranded overhang is a SP6 primer.

11. The method of synthesizing a DNA molecule of user-defined sequence of claim 1 wherein said single-stranded overhang is a three bases overhang.

12. The method of synthesizing a DNA molecule of user-defined sequence of claim 1 wherein said user-selected, single stranded DNA sequences are oligos of six bases.

13. The method of synthesizing a DNA molecule of user-defined sequence of claim 1 wherein said pre-defined, double-stranded, sequence of DNA and said preselected, single stranded DNA sequences are determined by using computational techniques to break said DNA molecule of user-defined sequence into fragments of defined size.

14. The method of synthesizing a DNA molecule of user-defined sequence of claim 1, including the step of ligating user-selected, single stranded DNA sequences to lengthen said surface-tethered, pre-defined, double-stranded, sequence of DNA using temperatures from 0 degrees C to 70 degrees C.

15. The method of synthesizing a DNA molecule of user-defined sequence of claim 1, including the steps of ligating user-selected, single stranded DNA sequences to lengthen said surface-tethered, pre-defined, double-stranded, sequence of DNA and using ligases comprising T4 DNA ligase.

16. The method of synthesizing a DNA molecule of user-defined sequence of claim 1, including the steps of ligating user-selected, single stranded DNA sequences to lengthen said surface-tethered, pre-defined, double-stranded, sequence of DNA and using ligases comprising T7 RNA ligase.

17. The method of synthesizing a DNA molecule of user-defined sequence of claim 1, including the steps of ligating user-selected, single stranded DNA sequences to lengthen said surface-tethered, pre-defined, double-stranded, sequence of DNA and using ligases comprising Ampligase thermostable DNA ligase.

18. The method of synthesizing a DNA molecule of user-defined sequence of claim 1, including the steps of ligating user-selected, single stranded DNA sequences to lengthen said surface-tethered, pre-defined, double-stranded, sequence of DNA and using ligases comprising T4 DNA ligase and/or T7 RNA ligase and/or Ampligase thermostable DNA ligase.

19. The method of synthesizing a DNA molecule of user-defined sequence of claim 1, wherein said steps of lengthening said pre-defined, double-stranded, sequences of DNA by the addition of user-selected, single stranded DNA sequences comprises sequentially combining reactions in which a first pre-defined sequence of DNA is lengthened by the addition of additional pre-defined sequences of DNA and sequentially combining reactions in which said first pre-defined sequence of DNA is lengthened by the addition of additional pre-defined sequences of DNA.

20. The A method of synthesizing a DNA molecule of user-defined sequence of claim 19 wherein said sequentially combining reactions in which a first pre-defined sequence of DNA is lengthened by the addition of additional pre-defined sequences of DNA and sequentially combining reactions in which said first pre-defined sequence of DNA is lengthened by the addition of additional pre-defined sequences of DNA are performed in parallel.

21. A method of-synthesizing a DNA molecule of user-defined sequence, comprising the steps of:
providing a first pre-defined sequence of DNA, sequentially combining reactions in which said first pre-defined sequence of DNA are lengthened by the addition of additional pre-defined sequences of DNA, and
sequentially combining reactions in which said additional pre-defined sequences of DNA are lengthened by the addition of yet additional pre-defined sequences of DNA.

22. The A method of synthesizing a DNA molecule of user-defined sequence of claim 21 wherein said steps of sequentially combining reactions in which said first pre-defined sequences of DNA are lengthened by the addition of additional pre-defined sequences of DNA and sequentially combining reactions in which said additional pre-defined sequences of DNA are lengthened by the addition of yet additional pre-defined sequences of DNA are performed in parallel.
